(19) United States
(12) Patent Application Publication
(10) Pub. No.: US 2018/0127342 A1
(43) Pub. Date: May 10, 2018

(54) EXTRACTION OF RESVERATROL FROM WASTE BIOMASS DERIVED FROM GRAPES PROCESSING

(71) Applicant: Kieu Hoang, Westlake Village, CA (US)

(72) Inventor: Kieu Hoang, Westlake Village, CA (US)

(21) Appl. No.: 15/672,124

(22) Filed: Aug. 8, 2017

Related U.S. Application Data
(60) Provisional application No. 62/371,883, filed on Aug. 8, 2016.

(51) Int. Cl. C07C 37/72; C07C 37/70

(52) U.S. Cl. CPC C07C 37/72; C07C 37/70

(57) ABSTRACT

The invention describes the extraction method to obtain the highest amount of resveratrol from wasted biomass produced by grapes processing. Resveratrol, which is found rich in the grapes, especially in grape skin and seed, is one of the most potent antioxidant, and can help to fight cell-damaging free radicals and promote cardiovascular health. Based on the most performing extraction methods to obtain resveratrol from plant material, the invention provides the optimized method of extracting the highest amount of resveratrol from wasted biomass from grapes processing.

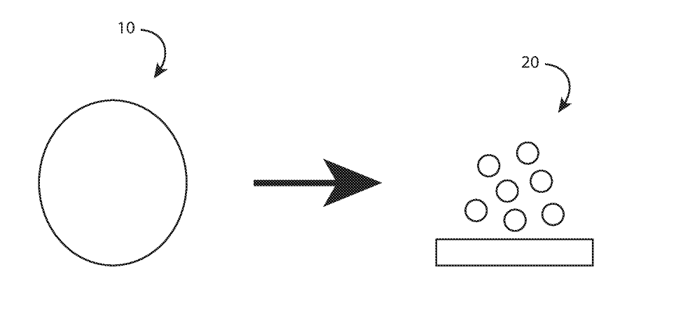

### CROSS REFERENCE TO PREVIOUS APPLICATION

This application claims the benefit of U.S. Provisional application 62/371,883, filed Aug. 8, 2016, which application is also incorporated herein by reference.

### REFERENCES CITED

1. Amparo Chafer, M. Carmen Pascual-Mart, Amparo Salvador, Angel Berna. Supercritical fluid extraction and HPLC determination of relevant polyphenolic compounds in grape skin. J. Sep. Sci., 2005, 28, 2050-2056.
2. Kashif Ghafoor, Jiyong Park, Yong-Hee Choi. Optimization of supercritical fluid extraction of bioactive compounds from grape (Vitis labrusca B.) peel by using response surface methodology. Innov. Food Sci. Emerg., 11 (2010) 485-490.
3. M. C. Pascual-Marti, A. Salvador, A. Chafer, A. Berna. Supercritical fluid extraction of resveratrol from grape skin of Vitis vinifera and determination by HPLC. Talanta 54(2001) 735-740.

### OTHER REFERENCES

1. Wagner H, Bladt S. Plant Drug Analysis, A Thin Layer Chromatography Altlas. 2009, second Edition, Springer.
2. Lopez Hernandez, J.; Paseiro Losada, P.; Sanches-Silva, A. T.; Lage-Yusty, M. A. Study of the changes of trans-resveratrol caused by ultraviolet light and determination of trans- and cis-resveratrol in Spanish white wines. Eur. Food Res. Technol. 2007, 225, 789-796.
3. Chaovanalikit, A.; Wrolstad, R. E. Total antocyanins and total phenolics of fresh and processed cherries and their antioxidant properties. Food Chem. Toxicol. 2004, 69, 67-72.
4. Scartezzini, P.; Antognoni, F.; Poli F.; Sabbioni, C. Vitamin C content and antioxidant activity of the fruit and of the ayurvedic preparation of Emblica officinalis Gaertn. J. Ethnopharmacol. 2005, 104, 113-118.
5. Casas L., C. Mantell, M. Rodriguez, E. J. Martinez de la Ossa, A. Roldán, I. De Ory, I. Caro, A. Blandino. Extraction of resveratrol from the pomace of Palomino fino grapes by supercritical carbon dioxide. Journal of Food Engineering 96 (2010) 304-308.
9. Gerogiannaki-Christopoulou Maria, Panagiotis Athanasopoulos, Nikolaos Kyriakidis, Ifigenia A. Gerogiannaki, Michalis Spanos. trans-Resveratrol in wines from the major Greek red and white grape varieties. Food Control 17 (2006) 700-706.
10. Feijòo Olalla, Anxela Moreno, Elena Falque. Content of trans- and cis-resveratrol in Galician white and red wines. Journal of Food Composition and Analysis 21 (2008) 608-613.
11. Cho Yong-Jin, Ji-Young Hong, Hyang Sook Chun, Sang Kook Lee, Hye-Young Min. Ultrasonication-assisted extraction of resveratrol from grapes. Journal of Food Engineering 77 (2006) 725-730.

### BACKGROUND OF THE INVENTION

Grape are popular foods and their ingredients are healthy for human. Resveratrol, as one of the most potent antioxidant, is found rich in the grapes, especially in grape skin and seed. Nowadays, a lot of waste biomass is produced during the grape processing. In fact, the waste parts of grape, such as skin, stem and seed, also contain much useful ingredients for human, like resveratrol. The target of our invention is to obtain the highest amount of resveratrol from wasted biomass produced by grapes processing.

### SUMMARY OF THE INVENTION

The invention provides a method to extract the highest amount of resveratrol from the waste biomass produced during grape processing. The invention enables the resveratrol, a valuable nutriment rich in the waste biomass containing grape skin and seed, to be extracted and used for human consumption. The value of grapes is maximized by this invention. The invention for obtaining juice and powder from the grape bunch, wherein the skin, seed,

### BRIEF DESCRIPTION OF THE DRAWINGS

The FIGURE shows a diagrammatic illustration of grounded biomass.

### DETAILED DESCRIPTION

The present invention describes an extraction method with the optimal performing conditions to obtain the highest amount of resveratrol from waste biomass produced by grapes processing. Supercritical fluid extraction (SFE) method is carried to extract.

The exhausted grapes are lyophilised using a lyophilizer LIO5P.

As shown in the FIGURE, the dehydrated sample 10 is then grounded in a variable-speed rotor mill Pulverisette 20 and stored at −20° C.

The supercritical fluid extraction is carried out using an Applied Separations extractor model Spe-Ed™ SFE Prime.

Just before the extraction, a 50 mL vessel is loaded with 6 g of powdered sample mixed with 4 g of Spe-Ed™ Matrix.

After the sample compression a plug of wool (Spe-Ed™ Wool) is place on top and the empty space is filled with matrix.

During the extraction process, the extraction CO₂ flow rate was maintained at an average level of 2 L/min.

7% to 10% of ethanol is used as co-solvents in supercritical fluids extractions.

The process is performed at 150 BAR, and the extraction temperature is 40-60° C. The process is performed for 1 min of static, followed by 15 to 20 min of dynamic extraction.

Extract is collected and stored at −20° C. for future use.

1. A extraction method to obtain the highest amount of resveratrol from the waste biomass produced by grapes processing.

2. The method of claim 1, wherein the extraction temperature is 40-60° C.

3. The method of claim 1, wherein 7% to 10% ethanol is used as co-solvent

4. The method of claim 1, wherein the extraction time of dynamic phase is between 15 to 20 min.
